(19) United States
(12) Patent Application Publication
(10) Pub. No.: US 2006/0257834 A1
(43) Pub. Date: Nov. 16, 2006

(54) Quantitative EEG as an identifier of learning modality

(76) Inventors: Linda M. Lee, Carmel, CA (US); Michael J. Lee, Carmel, CA (US); Hans C. Lee, Monterey, CA (US); Ilang M. Guiroy, Salinas, CA (US); Timmie T. Hong, Monterey, CA (US); William H. Williams, Monterey, CA (US)

Correspondence Address: Linda M. Lee, Sutie 226, 2600 Garden Road, Monterey, CA, 93940, US

(21) Appl. No.: 11/430,555

(22) Filed: May 9, 2006

Related U.S. Application Data
(60) Provisional application No. 60/679,635, filed on May 10, 2005.

(51) Int. Cl. G09B 19/00; A61B 5/04

(52) U.S. Cl. 434/236; 600/544

(57) ABSTRACT

A quantitative, objective method utilizing physiological data is provided for evaluating the modality of learning for a user and determining in which modality the user is most effective at learning. The method utilizes a session where the user is provided a set of information while wearing sensors for measuring physiological data such as brain electrical activity, and where the physiological data values are then correlated with learning modalities. The process can be performed quickly, without the bias and poor granularity of self-reported learning modality assessments. This method can be employed before the design of teaching curricula to ensure that learners are receiving information in the modality that suits them best and enables them to learn most effectively. This method may also be employed with a testing session to further correlate physiological data, learning modality, and testing performance.

### BACKGROUND OF THE INVENTION—PRIOR ART

A person's learning modality is defined as their preferred medium of receiving information. These sensory modes, originally defined by Walter Barbe and Raymond Swassing, are visual, auditory, and tactile/kinesthetic. Each one is preferred in varying degrees. Visual is learning by seeing, auditory is learning through hearing, and tactile/kinesthetic is learning by touching or doing. Willis and Hodson (1999) subdivided these even further. In the visual modality there are print and picture learners. Print learners learn best through the reading the written word; picture learners need to see an illustration in order to most easily comprehend or remember something. The auditory group is divided into listening and verbal. Listening learners simply need to hear it and verbal learners need to say it. The tactile/kinesthetic is separated into hands-on, whole body, sketching, and writing. The hands-on learners need to take things apart or touch them in some way. Whole body learners need to act or move. Sketching and writing learners absorb the material most effectively through drawing and writing, respectively.

Determining a user's most effective learning modality is of utmost importance. Success in learning may not rely only on how intrinsically effective users are at learning, but also on how successful the teaching methods are. Tests and examinations may objectively indicate if users have learned a topic, but this occurs only after the teaching rather than during the teaching process. There is no objective way by which learning and learning methods can be assessed in real time. Consequently, users who learn in a different manner than the modality utilized in teaching learn less and must expend more mental effort focusing on the material. In addition, quantitative methods do not exist to adapt the learning modality of users in real time.

Prior research has shown that when a user is using his or her most effective learning modality, he or she is requires less focus in order to absorb what is being taught (Carter, 1998). Behavioral indicators do not provide insights into when a user is mentally focused. For instance, users with hearing as a most effective modality may not be visually focused on a lecturer, but still have heard and processed the content of the lecture. In such a circumstance, however, the user is behaviorally diagnosed as not paying attention. Focus, orientation and arousal are the three elements of attention. Focus is brought about by a part of the thalamus which operates like a spotlight, turning to shine on the stimulus. Once locked, it shunts information about the target to the frontal lobes, which then lock on and maintain attention (Carter, 1998). When the frontal lobes are focusing and working hard, neurons create oscillations that can be measured by sensors such as an electroencephalogram (EEG).

Previous studies (Klimesch, 1999; Gevins, 1997; Mizuhara, 2004; Klimesch, 1994; Harmony, 2004) have shown that de-synchronization in EEG alpha frequencies (8-12 Hz) is positively correlated to cognitive performance and speed of processing information and are significantly higher in subjects with good memory. In addition, synchronization EEG theta frequencies (4-7 Hz) are related to the encoding of new information and to episodic memory.

Methods do not currently exist for objectively and quantitatively determining the methods and modalities in which a user learns most effectively. Information on learning modalities is almost exclusively found through qualitative assessments in which the user is asked to rank or choose modalities which the user likes or dislikes. Such information is qualitative, may suffer from bias, and indicate the preferred, rather than the most effective, learning modalities.

The works mentioned here as prior art have made progress in the insertion of quantitative methods into learning studies. However, they have so far failed to focus on how a user is learning, and instead have chosen to concentrate on determining if a user is learning at all. This is an important yet subtle distinction. The latter implies that the prior art in its current state cannot determine the user's efficiency at learning. This is the crucial next step that our invention provides.

### BACKGROUND OF INVENTION—ADVANTAGES

The important advantage of this invention is its exemplary ability to quickly, quantitatively, and objectively identify a user's most effective learning modality without the need for the user to self-report his or her preferred modality. The invention's quantitative measurements of the user learning in a variety of modalities enables modalities to be compared and ranked, progress over time to be tracked, testing performance to be correlated with modality and physiological data, in addition to many other uses. Our invention determines how the user is learning, which in turn provides educators with scientific information on why a user can excel in one subject and not another. This knowledge of a user's most effective learning modality is crucial for shaping the design of curricula and teaching methods.

Further advantages of our patent will become apparent from a consideration of the ensuing description and drawings.

### SUMMARY

This invention is a novel method for determining the learning modalities of a user based upon physiological data measured with sensors worn by the user. In the preferred embodiment, an EEG measures electrical activity of the brain and this data is provided as inputs into a formula. The formula determines the energy in the theta and alpha frequencies and computes mental focus as a ratio of theta frequency energy to alpha frequency energy.

### DRAWINGS—FIGURES

In the drawings, closely related figures have the same number but different alphabetic suffixes.

FIG. 1A illustrates a process flowchart for a method for determining a user's most effective learning modality where mental focus is calculated after all of the EEG data has been recorded.

FIG. 1B illustrates a process flowchart for a method for determining a user's most effective learning modality where mental focus is calculated in real time as new EEG data is received.

FIG. 2 illustrates in detail the steps involved in the calculation of mental focus.

FIG. 3 illustrates in detail the steps involved in the presentation of learning material to the user.

FIG. 4A illustrates in further detail the auditory learning modality.

FIG. 4V illustrates in further detail the visual learning modality.

FIG. 4T illustrates in further detail the tactile learning modality.

FIG. 5A illustrates a process flowchart for a method for determining and verifying a user's most effective learning modality that incorporates an additional testing phase where mental focus is calculated after all EEG data has been recorded and where the testing phase occurs sequentially after the user's most effective learning modality has been calculated.

FIG. 5B illustrates a process flowchart for a method for determining and verifying a user's most effective learning modality that incorporates an additional testing phase where mental focus is calculated after all EEG data has been recorded and where the testing phase occurs sequentially before the calculation of the user's most effective learning modality.

FIG. 5C illustrates a process flowchart for a method for determining and verifying a user's most effective learning modality that incorporates an additional testing phase where mental focus is calculated in real time as new EEG data is received and where the testing phase occurs sequentially after the user's most effective learning modality has been calculated.

FIG. 5D illustrates a process flowchart for a method for determining and verifying a user's most effective learning modality that incorporates an additional testing phase where mental focus is calculated in real time as new EEG data is received and where the testing phase occurs sequentially before the calculation of the user's most effective learning modality.

FIG. 6A illustrates a process flowchart for a method for determining a user's most effective learning modality that incorporates an additional display step where mental focus is calculated after all of the EEG data has been recorded.

FIG. 6B illustrates a process flowchart for a method for determining a user's most effective learning modality that incorporates an additional display step where mental focus is calculated in real time as new EEG data is received.

FIG. 7 illustrates an example of what is displayed during the display step.

FIG. 8 illustrates multiple examples of the correlation between required mental focus and testing performance.

FIG. 9 illustrates two embodiments of the formula used in the calculation of mental focus.

### DETAILED DESCRIPTION—PREFERRED EMBODIMENTS

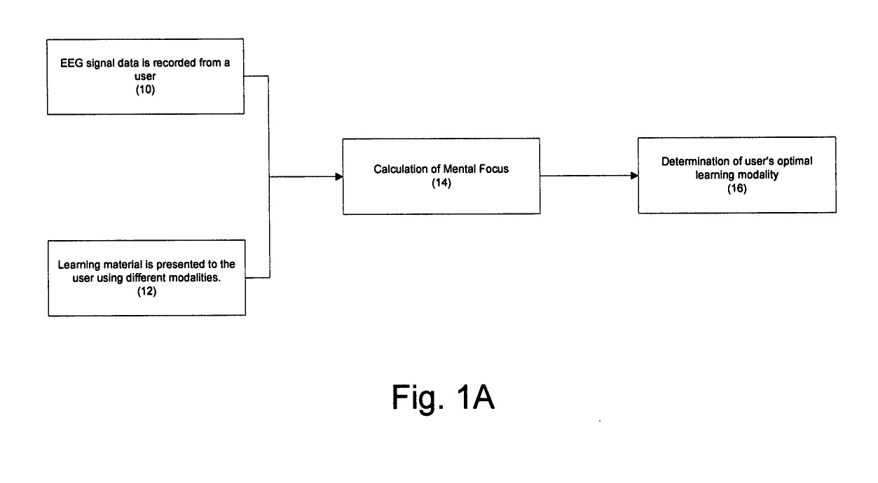

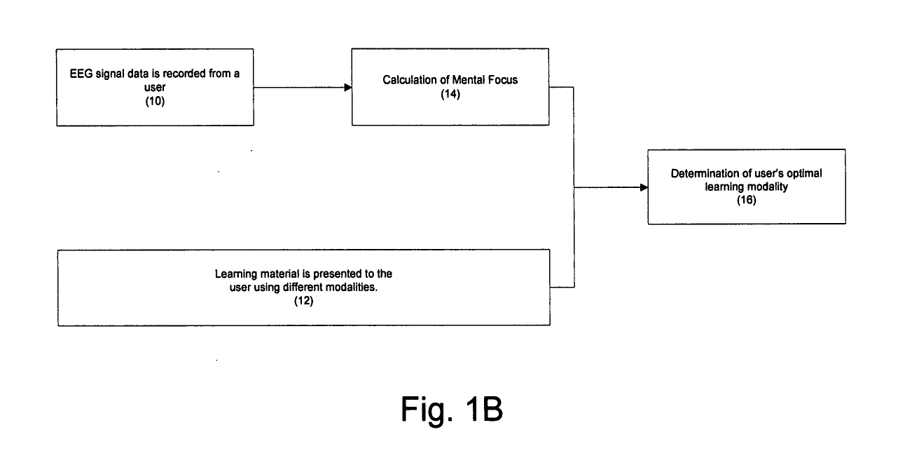

FIG. 1A represents a process flow for a method for determining a user's most effective learning modality. The user is presented 12 with learning material which engages each of several different learning modalities. At the same time EEG data is gathered 10 from the user, which in the preferred embodiment of our invention is done by having the user wear an EEG recording device. The electrical activity of the brain produced as the user learns the material is measured and recorded by the EEG. After all of the learning material has been presented, the user's mental focus is calculated 14 for each point in time during presentation process. Thereafter, the user's optimal learning modality is determined 16 by examining which learning modality in the learning phase 12 that required the lowest average mental focus, as calculated in mental effort calculation 14. In the preferred embodiment, the mental focus calculation 14 produces sixty data points per second of recorded EEG data. Such frequency allows for a sufficient granularity of mental focus so that a mental focus value or series of mental focus values can be associated with each learning modality.

FIG. 1B represents an alternative preferred embodiment of a method illustrated in FIG. 1A for determining a user's most effective learning modality where the mental focus of the user is calculated 14 in real-time sixty times per second as the user is learning the new material and new EEG data is received 10.

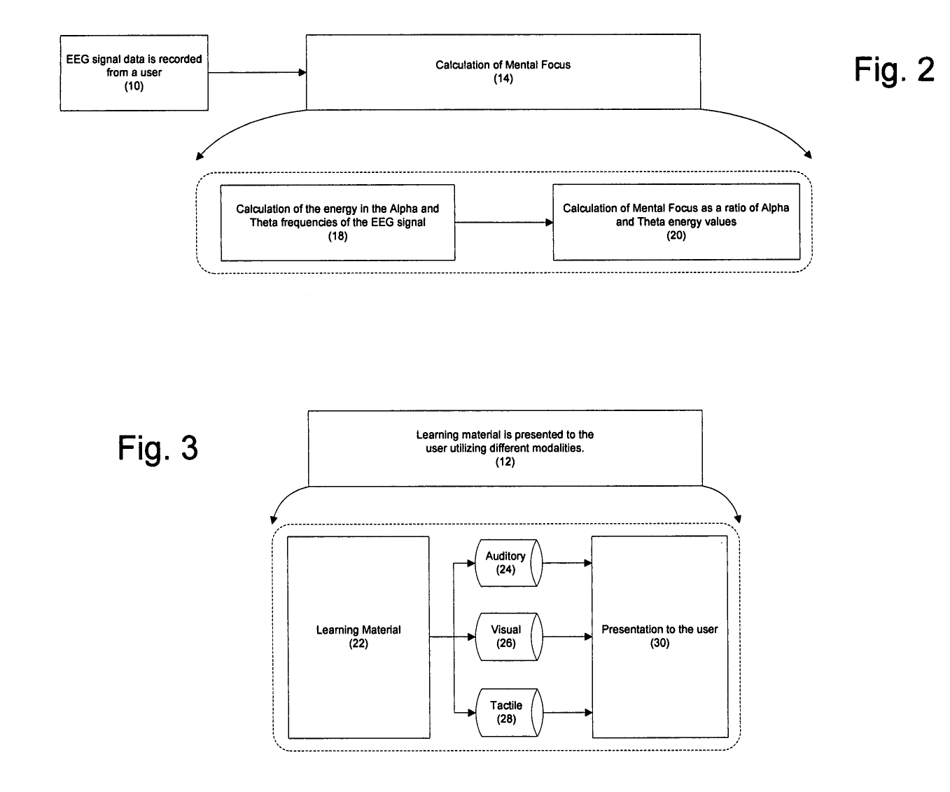

FIG. 2 shows further detail of the mental effort calculation 14 first described in relation to FIG. 1A. First the user's EEG data is measured 10. The two frequency bands of specific interest are Theta frequencies (between four and eight hertz) and Alpha frequencies (between eight and twelve hertz). After measurement 10, the energy of the Alpha frequencies and the energy of the Theta frequencies are calculated 18. This is accomplished by performing a Fourier Transform of the EEG data and examining the bins from one to twenty-four hertz. Then, the energy in each of the bins within the Theta band is summed and that value is defined as the Theta energy. The process for finding the Alpha energy value is very similar, with the value comprised of the sum of the energies of the individual bins within the Alpha band. Thereafter, the energies are used as inputs into a formula that determines a user's mental focus. In the presently preferred embodiment, the formula for the calculation 20 of mental focus is a ratio of Theta energy to Alpha energy.

FIG. 3 depicts the learning phase 12 first described in FIG. 1A in more detail. Learning material 22 is sequentially presented in a predetermined manner to the user utilizing an auditory modality 24, visual modality 26, and tactile modality 28. In the auditory modality 24, the user can hear the learning material or speak the learning material. In the visual modality 26, the user can read the learning material or view illustrations of the learning material. In the tactile modality 28, the user can write the learning material or draw the learning material. By hearing, speaking, reading, viewing, writing, or drawing the learning material 22, the user performs brain activity to retain the learning material 22 which enables the measurement of mental focus.

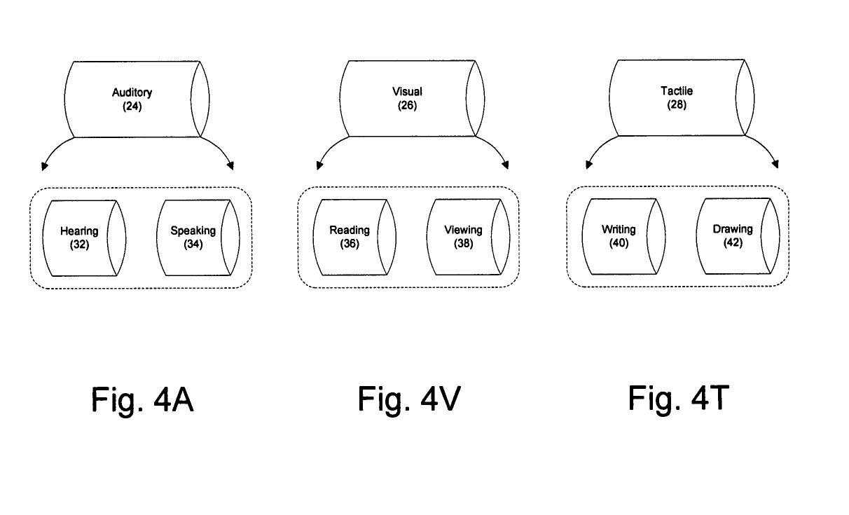

FIG. 4A shows in further detail the auditory modality 24. The auditory modality 24 of the learning phase 12 can comprise a hearing phase 32, where the user listens to a voice speaking the learning material. The auditory modality 24 may also comprise a speaking phase 34, where the user is instructed to repeat the presented learning material in his/her own voice aloud.

FIG. 4V shows in further detail the visual modality 26. The visual modality 26 of the learning phase 12 can comprise a reading phase 36, where the user reads the learning material as it is presented in written form. The visual modality 26 may also comprise a viewing phase 38, where the learning material is presented to the user in the form of drawings, illustrations, or pictures.

FIG. 4T shows in further detail the tactile modality 28. The tactile modality 28 of the learning phase 12 can comprise a writing phase 40, where the user writes the learning material, either on paper, on a computer, or gestures the information. The tactile modality 28 may also comprise a drawing phase 42, where the user is instructed to draw, graph, or otherwise illustrate the learning material.

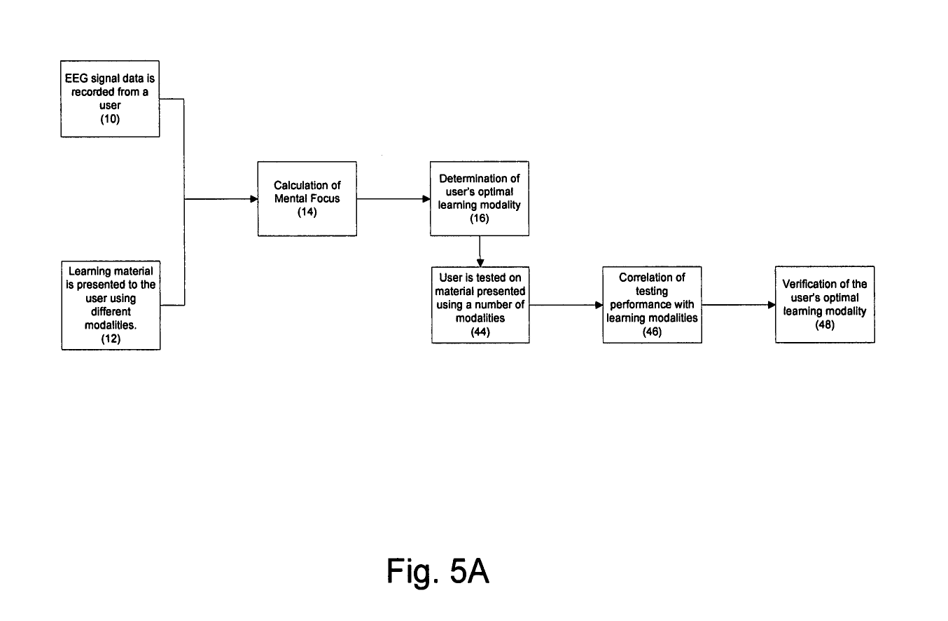

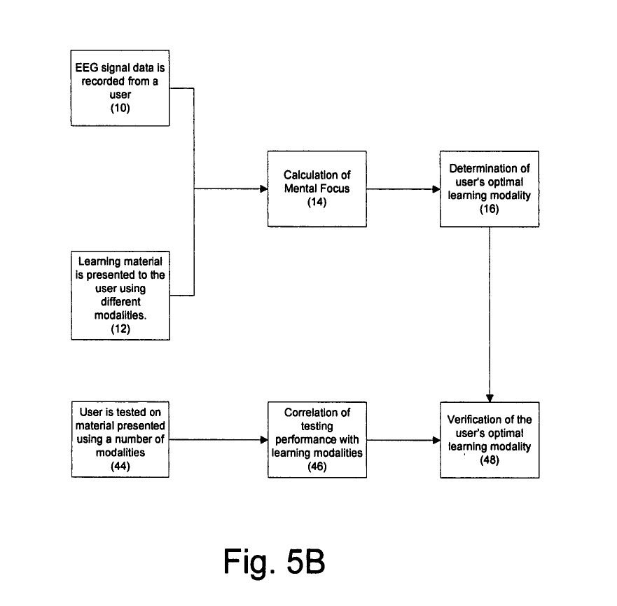

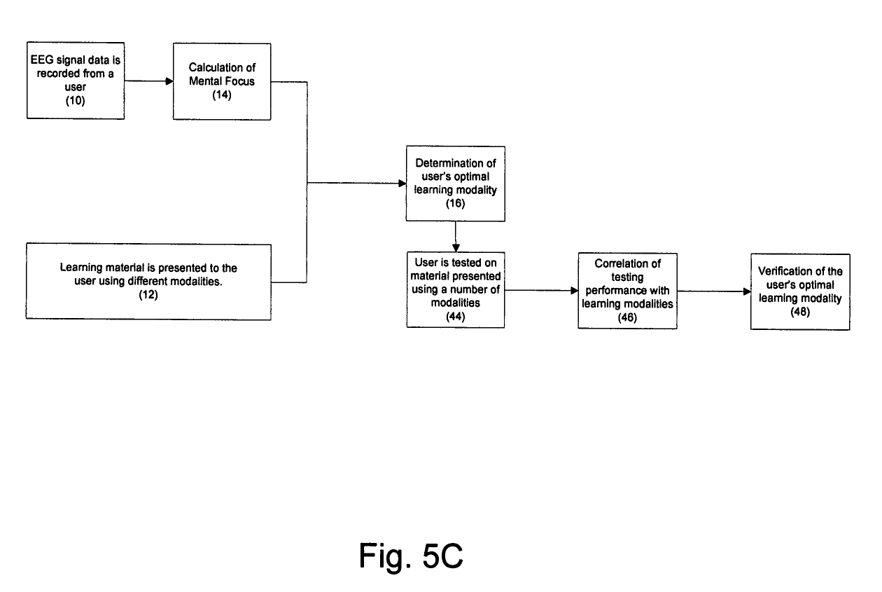

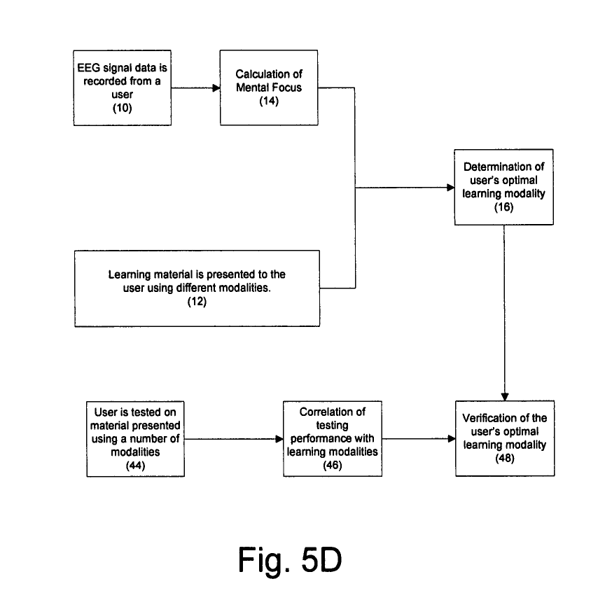

FIG. 5A depicts the method for the determination of a user's most effective learning modality first described in FIG. 1A and adds a further testing phase to the end of that process. After the determination of the user's optimal learning modality 16, the user is presented testing material in a number of different modalities, and then tested 44 on said material. Thereafter, testing performance, as measured by the number of questions answered correctly, is correlated 46 with the modality in which the associated information was presented in. After the correlation 46, verification that the user's optimal learning modality led to highest testing performance 48 can occur.

FIG. 5B depicts an alternative preferred embodiment of the combination of the method for the determination of a user's most effective learning modality first described in FIG. 1A and the additional testing phase first described in FIG. 5A. Instead of the testing phase occurring after the determination 16 of the user's most effective learning modality, the testing phase could occur before the learning material is presented 12 to the user. Thus the user is tested 44 on material and the user's scores are correlated 46 with learning modalities before the determination of the user's optimal learning modality. Verification 48 of the user's most effective learning modality does occur however after both processes are completed.

FIG. 5C depicts an alternative preferred embodiment where the method for the determination of a user's most effective learning modality first described in FIG. 1B is combined with an additional testing phase in the same manner as described in FIG. 5A.

FIG. 5D depicts an alternative preferred embodiment where the method for the determination of a user's most effective learning modality first described in FIG. 1B is combined with an additional testing phase in the same manner as described in FIG. 5B.

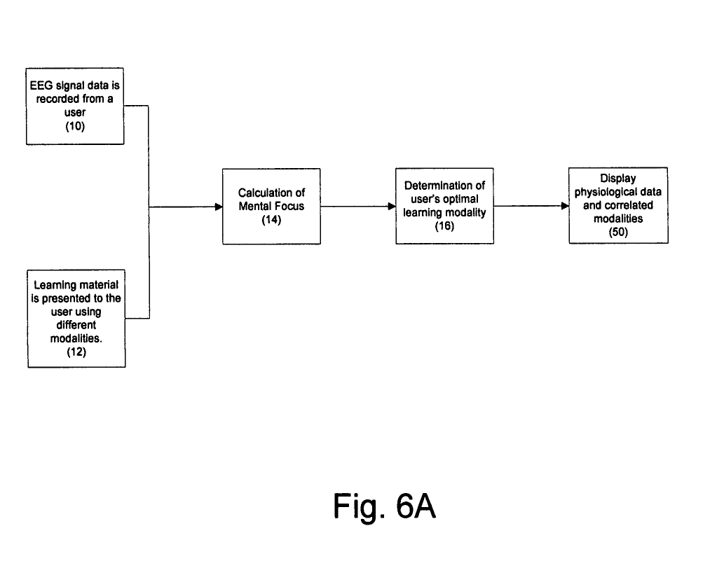

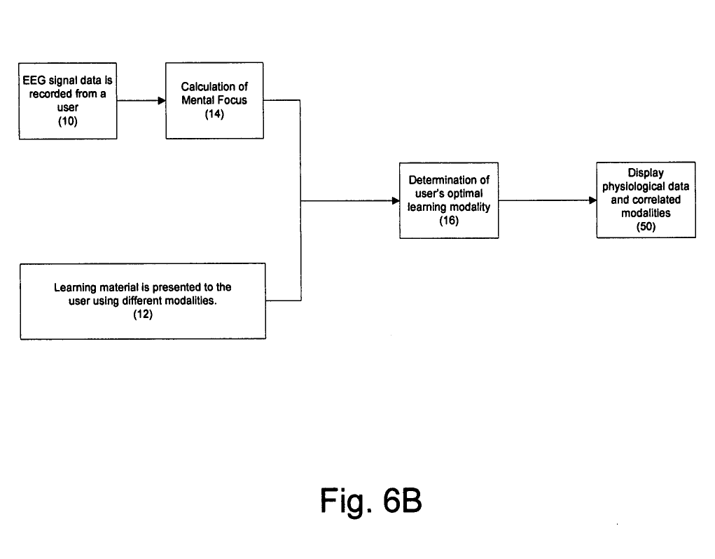

FIG. 6A depicts the process flow of FIG. 1A and adds a further display phase 50. In the preferred embodiment, the display phase 50 displays on a computer screen information collected during the mental focus calculation 14 and learning phase 12, comprising, in particular, the mental focus and the average level of mental focus during the learning modality in a graph form, allowing for easy viewing of the mental focus measured during each learning modality.

FIG. 6B depicts an alternative preferred embodiment that takes the process flow described in FIG. 1B and adds a further display phase 50 in the manner described in FIG. 6A.

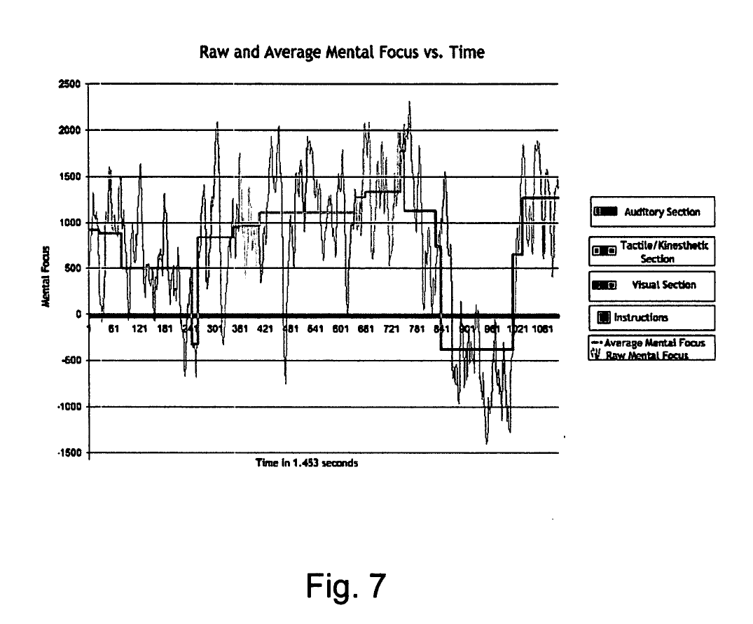

FIG. 7 illustrates an example of the display phase 50. The calculated value of mental focus is depicted on the y-axis, while time occupies the x-axis. Line segments denote the average value of mental effort for each modality section. The full mental focus plot is also indicated, demonstrating the granularity of the measurement 10 of physiological data. In this particular example, the mental focus during the visual modality section is substantially lower, indicating that the user expended less mental effort in order to focus while learning in this section and that the visual modality would be this user's most effective modality.

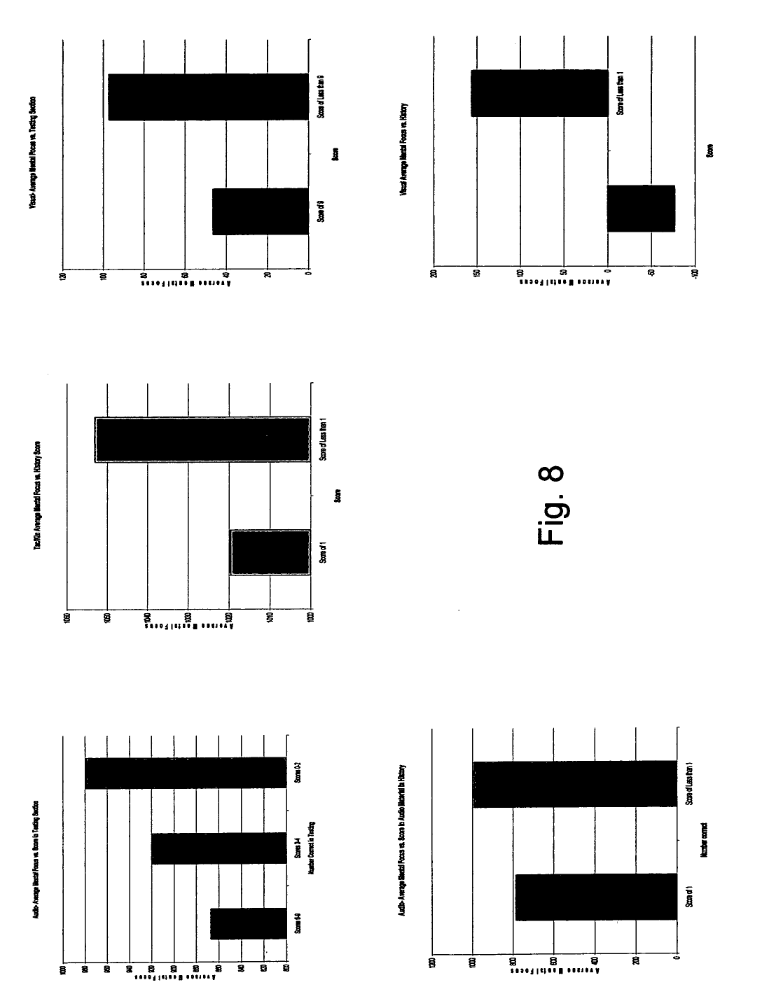

FIG. 8 illustrates the correlation between mental effort and testing performance. Each column of bar graphs is associated with the mental focus and testing performance of a single learning modality. Each bar indicates the average mental focus across all users who scored in a particular score range. For all three modality types, users who scored higher in the testing phase exhibited, on average, lower mental focus. This demonstrates that determining a user's most effective learning modality in an objective and quantitative manner is a reality.

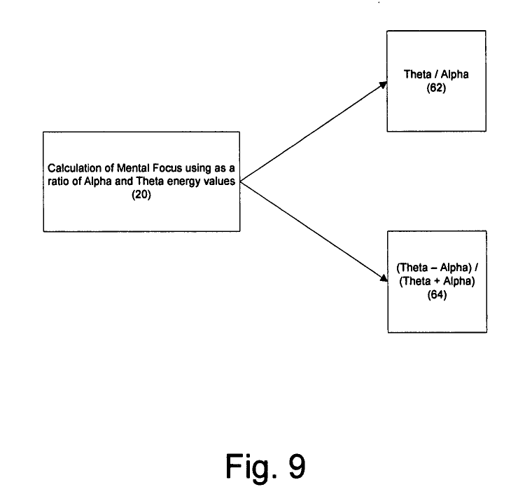

FIG. 9 depicts two embodiments of the formula used in the calculation of mental effort 20. One embodiment is the ratio of Theta energy and Alpha energy 62. With Theta in the numerator and Alpha in the denominator of this ratio, the formula is an indicator of mental focus. Lower values of this formula indicate that less mental effort is being expended by the user in order to focus, and thus the learning material 22 is being absorbed more easily. However, one could also construe a formula with Alpha in the numerator and Theta in the denominator of the ratio. Such a formula would exhibit higher values when learning material 22 is being absorbed more easily, and thus would correspond to an indicator of mental ease. Another embodiment 64 of the formula is the difference between Theta energy and Alpha energy, this value then divided by the sum of Theta energy and Alpha energy. This corresponds to a mental focus formula, where lower values indicate that learning material 22 is being absorbed more easily. In both embodiments, higher Theta energy indicates higher cognitive activity while higher Alpha energy indicates lower cognitive activity.

### CONCLUSION, RAMIFICATIONS, AND SCOPE

Thus, the reader can see that this method for determining a user's most effective learning modality is quick, quantitative, and objective, and will enable a leap forward in the development of education curricula.

While the above description contains many specificities, these should not be construed as limitations on the scope of the invention, but rather as an exemplification of one preferred embodiment thereof. Many other variations are possible. For example:
The use of a different calculation rate other than 60 Hz.
Other known and yet undiscovered learning modalities may be utilized.
The order of the modalities in which the learning material is presented may be different than as described in the preferred embodiment.
Data may be displayed on any type of display device, in place of a computer screen.
The display of data may take a form different than that of a graph.
Any term described in the formula for the calculation of mental focus may be generalized. For example, if we let the variable x represent the current amount of Theta energy, then any value of the form a*x+b where a and b are constants would be correct to insert into the formula in place of x.
The method described could also be used to discover and test new possible learning modalities, by understanding their similarities and complexities to those modalities that are currently known.

Accordingly, the scope of our invention should be determined not by the embodiments illustrated, but by the appended claims and their legal equivalents.

We claim:

1. A method for determining a user's most effective learning modality comprising:
a. measuring physiological data of a user with a group comprising of a single or plurality of sensors,
b. when measuring the user, providing a learning phase, where a set of information is provided to the user in a predetermined number of different modalities,
c. correlating the physiological data with the modalities in the learning phase,
d. comparing the physiological data of the modalities, whereby the user's most effective learning modality can be determined quickly, quantitatively, and objectively.

2. A method of claim 1 wherein the sensors comprises a single or plurality of electroencephalogram sensors.

3. A method of claim 2 wherein the physiological data comprises electrical activity of the brain of the user.

4. A method of claim 3 wherein the electrical activity comprises alpha and theta frequencies.

5. A method of claim 4 further comprising:
a. calculating an alpha energy using said alpha frequencies and calculating a theta energy using said theta frequencies,
b. utilizing the alpha energy and the theta energy as inputs in a formula, where said formula is a measure of mental effort, where the mental effort is used in place of the physiological data for correlating and comparing.

6. The method of claim 5 wherein the formula comprises a ratio of alpha energy and theta energy

7. The method of claim 1 wherein said modalities comprises an auditory learning modality.

8. The method of claim 7 wherein the set of information provided in the auditory learning modality comprises the user hearing the set of information.

9. The method of claim 7 wherein the set of information provided in the auditory learning modality comprises the user speaking the set of information.

10. The method of claim 1 wherein said modalities comprises a visual learning modality.

11. The method of claim 10 wherein the set of information provided in the visual learning modality comprises the user reading the set of information.

12. The method of claim 10 wherein the set of information provided in the visual learning modality comprises the user viewing illustrations of the set of information.

13. The method of claim 1 wherein said modalities comprises a tactile learning modality.

14. The method of claim 13 where the set of information provided in the tactile learning modality comprises the user writing the set of information.

15. The method of claim 13 where the set of information provided in the tactile learning modality comprises the user drawing the set of information.

16. The method of claim 1 further comprising:
a. providing a testing phase where the user is tested on the set of information provided in the learning phase, whereby the testing phase serves to verify the most effective learning modality and correlate the mental effort of each modality with testing performance

17. The method of claim 1 further comprising:
a providing a display for displaying the physiological data and correlated modalities.

18. The method of claim 1 further comprising:
a. utilizing the most effective learning modality in a curriculum for teaching a topic, whereby said curriculum is more effective at teaching said topic because the user learns in the most effective modality

19. The method of claim 18 wherein said curriculum is presented as an interactive video game

20. The method of claim 1 further comprising:
a. saving to a computer readable storage media the physiological data and correlated modalities

21. The method of claim 1 further comprising:
a. repeating (a)-(c) of claim 1 an indeterminate number of times over a period of time to obtain a set of physiological data and correlated modalities,
b. comparing the set of physiological data and correlated modalities

22. The method of claim 1 further comprising:
a. recording the user's demographic information
b. repeating (a)-(c) of claim 1 and (a) of claim 22 for an indeterminate number of users to obtain a set of physiological data and correlated modalities
c. comparing the set of physiological data and correlated modalities and demographic information

23. The method of claim 1 further comprising:
a. ranking the effectiveness of different modalities according to the physiological data
